(19) United States
(12) Patent Application Publication
(10) Pub. No.: US 2005/0065532 A1
(43) Pub. Date: Mar. 24, 2005

(54) Surgical instruments

(76) Inventors: Matthias Honl, Norderstedt (DE); James Brooks, Burley-in-Wharfedale (GB); Stefano Alfonsi, Marrogate (GB); Gary Moore, West Yorkshire (GB)

Correspondence Address: Paul J Maginot;Maginot Moore & Beck, Bank One Center Tower, 111 Monument Circle Suite 3000, Indianapolis, IN, 46204-5115, US

(21) Appl. No.: 10/490,792

(22) PCT Filed: Sep. 25, 2002
(86) PCT No.: PCT/GB02/04334

(30) Foreign Application Priority Data
Sep. 27, 2001 (GB) 0123257.8
Nov. 3, 2001 (GB) 0126437.3

(51) Int. Cl. A61B 17/58

(52) U.S. Cl. 606/96

(57) ABSTRACT

An instrument for use in orthopaedic surgery for location of a reference point relative to a bone surface comprises a plate member (30) having at least two holes (36, 38) in it towards one end for receiving fasteners which can pass through the holes into the bone to fasten the plate member to the bone. A pivot rocker (40) is provided between the said holes, which protrudes from the face of the plate member which faces the bone surface when the plate member is positioned against a bone, and a reference point (44) is provided towards the opposite end of the plate member form the fastening holes which is spaced from the fastening holes in a direction parallel to the line which intersects the holes. The distance between the reference point on the plate member and the surface of the bone can be adjusted by moving the fastener in one of the fastening holes into the bone and moving the fastener in the other of the fastening holes out of the bone.

This invention relates to instruments for use in orthopaedic surgery, including an instrument for location of a reference point relative to a bone surface, and a connector for location of a surgical instrument relative to a reference point. Instruments are used in orthopaedic surgery for location of instruments such as drills, saws, reamers and the like, to optimise the accuracy of subsequent steps in a surgical procedure using those instruments. For example, an instrument can be used to define an axis relative to a bone, along which a cavity in the bone is subsequently prepared using tools such as drills and reamers. Instruments for use in such location steps should enable satisfactory accuracy to be achieved. They should minimise trauma to the patient and, in particular, minimise disturbance of tissue which would not otherwise be affected by the surgical procedure.

U.S. Pat. No. 5,766,263 and U.S. Pat. No. 6,120,544 relate to a femoral components of artificial hip joints which are mounted in the upper region of the femur below the greater trochanter, within and aligned with the femoral neck axis. These components can be contrasted with widely used hip joint femoral components which extend into the intramedullary cavity extending along the femur axis. Use of the components with which U.S. Pat. No. 5,766,263 and U.S. Pat. No. 6,120,544 are concerned has the advantage that less of the patient's natural bone tissue is removed during surgery: the bone is resected on the femoral neck, just below the spherical head, rather than by removal of the entire head section of the femur, including the greater trochanter by resection in the metaphysial region. In addition, the length of the incision that is required is smaller than with conventional implants. The procedure for implanting a prosthesis of the kind disclosed in U.S. Pat. No. 5,766,263 and U.S. Pat. No. 6,120,544 is therefore less traumatic for the patient. The resection of less bone allows better for the possibility of revision surgery at a later date in the event that the original prosthesis implantation fails.

The prostheses disclosed in U.S. Pat. No. 5,766,263 and U.S. Pat. No. 6,120,544 are implanted on the axis of the femoral neck. Preparing the bone for the implantation requires accurate location of the neck axis. This is also important because the resection plane should generally be orthogonal to this axis. The axis can be defined by locating a point on the lateral cortex which lies opposite to the centre of the femoral head, where the axis intersects the cortex. This can be identified during pre-operative planning, for example using X-ray or other images of the femur, in particular relative to prominent landmarks on the bone surface. In the case of the prosthesis disclosed in U.S. Pat. No. 5,766,263, a hole is formed in the lateral cortical tissue for a draw-in screw, by which a shell part introduced into the femur through the resected femoral neck is drawn into the bone. The hole can be formed by a drill after resection of the bone, extending through the femur, along the neck axis. Drill guides which enable accurate location of the neck axis for drilling from the lateral surface to the medial surface are known. They have the disadvantage that they require exposure of the point on the lateral cortical bone at which the axis intersects that bone, involving in particular the displacement of muscle and other soft tissue.

In the case of the prosthesis disclosed in U.S. Pat. No. 6,120,544, the femur remains intact in the region of the lateral cortex opposite to the resected femoral neck. The prosthesis is inserted into a prepared cavity in the bone, and acts against the lateral cortical bone tissue, and the fact that the tissue remains intact helps it to withstand the loads transmitted on to it when the prosthesis is placed under load. However, there remains a need to locate the neck axis accurately when preparing the cavity for implantation of a femoral component.

In one aspect, the invention provides an instrument for use in orthopaedic surgery for location of a reference point relative to a bone surface, which comprises a plate member having at least two holes in it towards one end for receiving fasteners which can pass through the holes into the bone to fasten the plate member to the bone, a pivot rocker between the said holes which protrudes from the face of the plate member which faces the bone surface when the plate member is positioned against a bone, and a reference point towards the opposite end of the plate member from the fastening holes in a direction parallel to the line which intersects the holes, in which the distance between the reference point on the plate member and the surface of the bone can be adjusted by moving the fastener in one of the fastening holes into the bone and moving the fastener in the other of the fastening holes out of the bone.

The instrument of the invention has the advantage that it allows a reference point to be located relative to a bone surface using a plate member which is fastened to the bone remote from the reference point. In particular, the nature of the fixation of the plate member to the bone surface using two (or more) fixation holes with a pivot rocker between them means that the distance between the plate and the bone at the reference point can be adjusted. This can be important when it is important for the reference point to be located as close as possible to the bone surface, for example when defining an axis which passes through the bone surface at a previously identified location (corresponding as closely as possible to the location of the reference point on the plate member).

The instrument of the invention therefore allows a reference point to be established relative to a bone surface without direct access to the bone surface at that point, for example for fastening the instrument to the bone. This can be important when soft tissue, especially muscle tissue, overlies the bone surface at the intended reference point which would have to be displaced if the plate member is to be fastened to the bone surface using fasteners inserted into the bone at or close to the reference point. The instrument of the invention therefore provides for location of a reference point accurately and closely relative to a bone surface, and fixation to the bone surface, without disturbing soft tissue which overlies the bone at the reference point.

The location of the plate member relative to the bone can be performed using data from pre-operatively generated images, in particular by identifying appropriate landmarks on the bone surface relative to the appropriate location of the plate member.

Preferably, the instrument includes a connector rod for another component, which is pivotally connected to the plate member at the reference point. For example, the connector rod can be used for mounting an instrument which is used during preparation of the bone for implantation of a prosthesis component. Examples of such instruments include drills, saws, reamers and the like. The instrument might also be a locator for a sleeve which is fastened to the resected femoral neck to define the neck axis. The instrument of the present invention allows the orientation of such instruments to be controlled relative to the reference point defined by the plate member.

Preferably, the relative pivotal motion of the connector rod relative to the plate member is such that the connector rod can be pivoted about an axis which extends roughly parallel to the axis of the bone. This can have considerable advantages in terms of minimising the size of the incision that is necessary during preparation of a bone for implantation of a prosthesis. For example, the instrument of the present invention can be fastened to a bone laterally using the fastening holes in the plate member, with the connector rod located outside the incision, and the region of the bone that is exposed including only (a) the intended location of the reference point, and (b) the bone surface into which the fasteners (for example screws) are to be inserted. An axis which includes the reference point can then be defined by rotating the bone around its axis relative to the incised soft tissue, pivoting the connector rod relative to the affixed plate member.

Preferably, the connector rod has an arcuate shape so that it can extend around the bone from the bone surface against which the reference point is located to an opposite bone surface to define an axis which extends between the two surfaces.

Preferably, the connection between the connector rod and the plate member permits relative pivotal motion about at least two axes, and possibly about three axes for some applications. When the connector rod and plate member can pivot about two axes, they will often be orthogonal. However, other arrangements might be appropriate for some applications.

The connector rod can include a mount at its end remote from the connection to the plate member for another instrument component, for example such as a saw, reamer or drill. When the connection between the connector rod and the plate member permits relative pivotal motion about at least two axes, the mount for the other instrument component can allow the component to be pivoted relative to the connector rod around an axis which is orthogonal to each of the axes of rotation of the connector rod relative to the plate member.

The mount can includes a track on which the surgical instrument can slide relative to the connector rod, so that the connector rod can be moved pivotally relative to the reference point without a significant change in the position of the instrument. Preferably, the track is arcuate, especially with a radius approximately equal to the distance from the track to the reference point on the plate member. This allows the connector rod to be moved pivotally relative to the plate member so that the connector rod can be fitted close to the bone to which the plate member is fastened. This has the significant advantage of enabling the size of the incision to be kept small while accommodating the instrument during the procedure, and also enabling the instrument to be used on patients with relatively bones.

It can be preferred for the connector rod to comprise at least two modules, so that the configuration of the connector rod can be changed by replacement of one of the modules. For example, one module can be provided in a plurality of variants, for example with different lengths, allowing the length of the connector rod to be changed.

The connector rod can carry a scale for indicating where the bone should be resected. The rod might have a cutting edge to facilitate marking the bone. A scale can also be provided on a module of a connector rod which can be replaced by another module after the location of the resection has been determined.

The reference point has been discussed above as the point at which a connector rod is connected to the plate member. It could be marked on the plate member in other ways. For example, the plate member could have a guide hole at the reference point, for example for receiving a drill by which a hole can be formed in the bone at the reference point. It might also be marked in other ways such as by the location of a marker which is designed to be visible using an appropriate scanning technology. For example, a tantalum marker can be used to locate a reference point so that it is visible using X-ray scanning technology.

The pivot rocker protrudes from the face of the plate member which faces the bone surface when the plate member is positioned against a bone, at a point between the holes for the fasteners. The rocker will often be located so that the pivot point is roughly equidistant from the two holes. The rocker can be configured so that it defines a ridge extending roughly perpendicular to a line which intersects the two fastening holes, and generally parallel to the plane of the plate member (when it is planar). The height of the ridge will depend on, amongst other things, the shape of the bone on which the plate is to be used, the distances between the pivot rocker and the fixation holes, and the distance between the pivot rocker and the reference point. For some applications, the height of a suitable ridge will be at least about 2 mm, preferably at least about 4 mm. The distance between the fixation holes can be at least about 15 mm, preferably at least about 25 mm. The distance from the pivot rocker to the reference point can be at least about 25 mm, preferably at least about 30 mm. The distance from the pivot rocker to the reference point can be not more than about 40 mm, preferably not more than about 35 mm.

The fixation holes can be countersunk. This has the advantage that fasteners such as screws can be recessed within the holes, reducing the risk of damage to tissue when the device is moved relative to overlying tissue.

Preferably, the reference point lies on the line which intersects the fastening holes. However, for some configurations of plate members, the reference point will be spaced from the fastening holes in a direction parallel to that line, but displaced from that line to one side or other. Preferably, the distance between the fastening holes is less than the distance between the reference point and the fastening hole that is nearer to it, measured along the line which intersects the fastening holes or a line extending parallel to it.

The plate member will be configured having regard to the configuration of the bone surface on which it is to be used. When the bone surface is generally flat (at least over that part with which the plate member comes into contact), the plate member can be generally flat, apart of course from the pivot rocker. However, the bone surface (together with any associated overlying soft tissue) will often not be flat. For example, when the invention is intended to be used to locate the lateral end of the femoral neck axis, the relevant lateral surface of the bone has an overlying layer of muscle. It is also concave. It can therefore be appropriate for the plate member to have an arcuate configuration so that it generally follows the shape of the bone on to which it is to be fitted when the instrument is in use.

Embodiments of the present invention will now be described by way of example with reference to the accompanying drawings, in which:

FIG. 1 is an isometric view of a known instrument which is used to define the neck axis of a femur in order to prepare the femur for implantation of a prosthetic joint component.

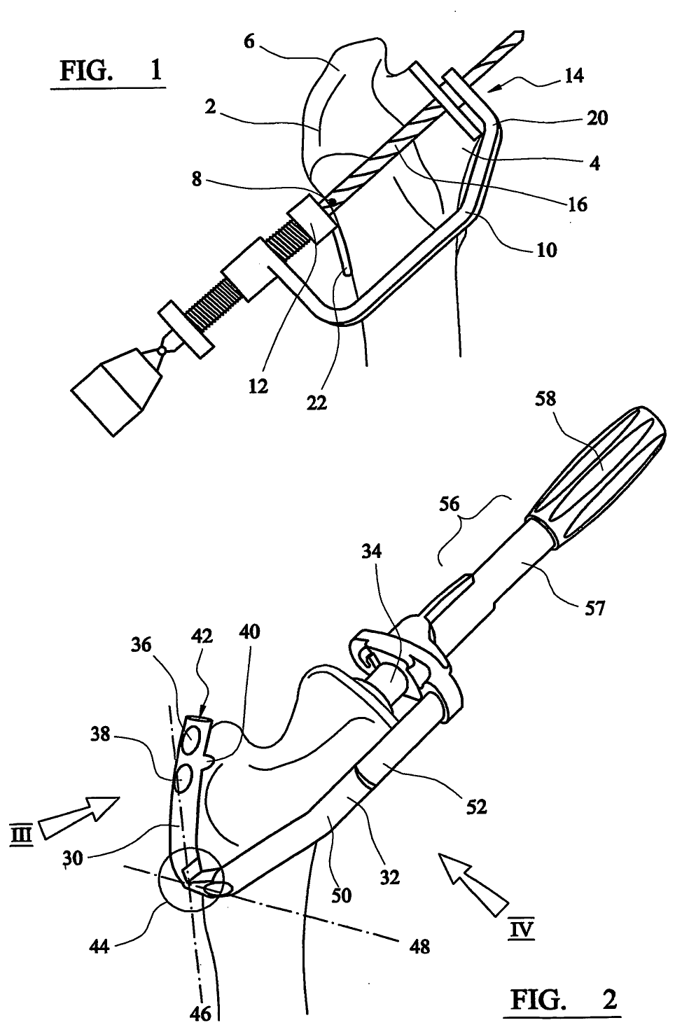

FIG. 2 is an isometric view of an instrument according to the present invention.

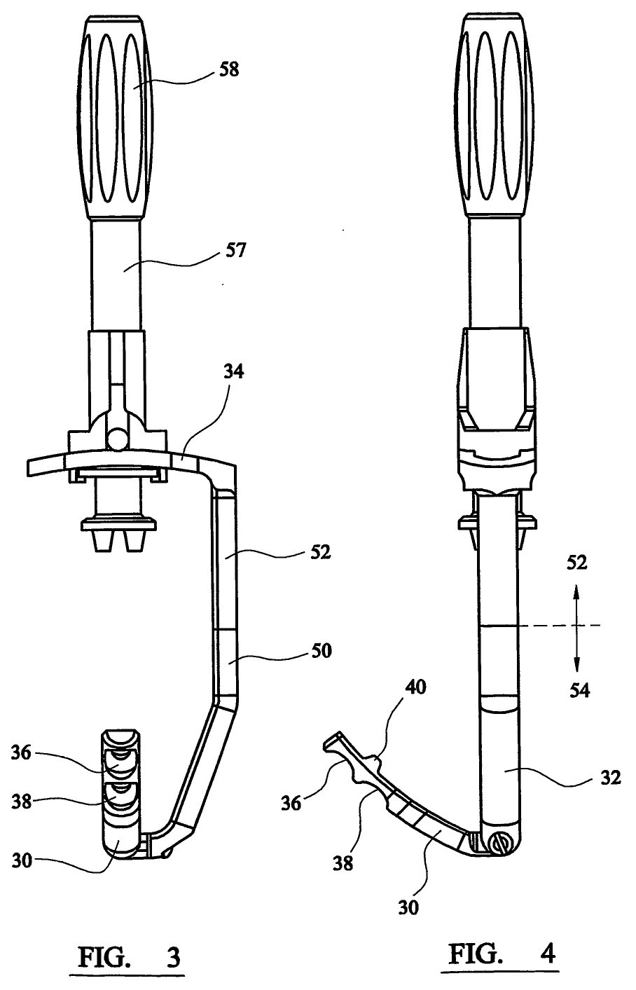

FIG. 3 is a side view of the instrument shown in FIG. 2, along the line indicated by the arrow III.

FIG. 4 is an end view of the instrument shown in FIG. 2, along the line indicated by the arrow IV.

Referring to the drawings, FIG. 1 shows a femur 2 which has been resected in the neck region 4 below the spherical head. The greater trochanter 6 remains intact as a result of this resection.

The femoral neck has to be prepared by creation of a cavity along the axis of the neck, into which the femoral of a hip joint prosthesis can be inserted. The axis is located by determining the point 8 on the lateral cortical bone tissue where the axis intersects the cortical bone. This can be done with reference to pre-operation scan images of the patient's bone. A drill guide 10 can be fixed relative to the point 8 on the cortex using a sleeve 12 which is fastened to the bone. The other end 14 of the axis is determined by visual inspection of the resected femur.

The instrument shown in FIG. 1 can then be used to prepare an axial bore, using a drill 16 in conjunction with the drill guide 10. One arm 20 of the drill guide is positioned against the resected femur, and the drill guide extends around the bone to intersect the axis on the lateral side of the bone where it engages the sleeve 12, allowing the guide then to be used to locate the drill as it is inserted into the bone tissue.

The drill guide arrangement shown in FIG. 1 has a number of disadvantages. In particular, a large incision is required in order to expose the bone sufficiently. In addition, in order to fasten the sleeve 12 onto the lateral cortical bone by means of a fixation plate 22, and also to drill into that bone, it is necessary to strip away muscle and other tissue which overlies the bone in this region. This complicates the surgical procedure and can delay or prevent full recovery by the patient.

The device of the present invention is shown in FIGS. 2 to 4. It comprises a plate member 30, a connector rod 32, and a mount 34 for a surgical instrument. The plate has two fastening holes 36, 38 towards one end, with a pivot rocker 40 protruding from the surface 42 which faces towards the bone when the device is in use. The plate member is slightly curved so that it can follow the concave shape of the femur on the lateral face below the greater trochanter.

The plate member is connected by means of a joint to the connector rod 32 at the end of the plate member 30 opposite to the fastening holes. The connection is made by means of a universal joint 44 which allows the connecting rod to be pivoted relative to the plate member about two orthogonal axes 46, 48. One of those axes 46 is aligned approximately with the axis of the femur, at least so that the connector rod can be pivoted around the femoral axis.

The connector rod 32 is provided in two modules 50, 52. The first module 50 is connected to the plate member by means of the universal joint. The second module 52 can be replaced by other second modules with different lengths in order to vary the effective rod of the connector rod. The connection between the modules can be provided by means of a press-fit, spigot and socket arrangement, for example with a spring loaded stud and detent arrangement to prevent inadvertent separation. The second module might have other functions other than to bear a instrument mount. For example, it might include a scale to enable a surgeon to judge where the bone should be resected for receiving the implant. The module might have a cutting edge to facilitate marking the bone. A scale can also be provided on a connector rod which is not modular.

The connector rod 32 has a mount 34 at the end remote from the plate member for mounting a surgical instrument such as a drill or a reamer, or a manipulator 56 (as shown) for a sleeve 57 which will define the femoral neck axis, and into which a drill can be inserted to create the neck bore. The manipulator 56 includes a handle 58. The sleeve with the overlying handle are hollow so that a drill or reamer or other tool can be inserted into the sleeve. The mount is arranged on the connector rod so that it can be pivoted relative to the end of the rod. The axis about which the mount can be pivoted is orthogonal to the two axes 46, 48 about which the connecting rod can be pivoted relative to the plate member.

The mount 34 comprises a track which the sleeve 57 can be slid along, along a line in the plane of the page on which FIG. 3 is shown. The track is arcuate with a radius approximately equal to the distance from the track to the reference point on the plate member. This allows the connector rod 32 to be moved pivotally relative to the plate member so that the connector rod can be fitted close to the bone to which the plate member is fastened. This has the significant advantage of enabling the size of the incision to be kept small while accommodating the instrument during the procedure, and also enabling the instrument to be used on patients with relatively bones.

In use, a patient's femur is prepared by incision to expose the femur in the region of the lateral cortex. It is possible for the length of the incision to be kept as low as about 60 mm. Previously generated scanned images enable the location of the intersection of the femoral neck axis with the lateral cortex (the “target point”) to be identified. The plate member 30 is then fixed to the femur at a location remote from the target point, in a direction towards the greater trochanter. It is fixed by means of bone screws which are inserted into the femur through the fixation holes 36, 38. The tension that is applied to the screws is controlled so that the universal joint 44 at the opposite end of the plate member is located as close as possible to the target point on the lateral cortex. It will be understood that the bone itself in this region is covered with muscle and other tissue. The provision of the pivot rocker with a fixation screw on each side allows the orientation of the plate member relative to the bone surface to be adjusted so that the universal joint end of the plate member is located as close as possible to the muscle. In contrast with the known device shown in FIG. 1, it is not necessary with the device of the invention to expose the bone at the target point in order to achieve appropriate fixation of the instrument relative to the target point.

The bone can then be rotated relative to the site of the incision, feeding the connector rod 50 around the bone so that, when the resected femoral neck is exposed, the guide arm extends around the bone and the sleeve 57 is mounted above the resected neck. The orientation of the sleeve (degree of anteversion) can be adjusted by manipulation of the mount relative to the connector rod, according to the surgeon's judgment. The mount can include a scale to indicate the degree of anteversion that is applied to the instrument. The sleeve can then be used to prepare a cavity in the femur for the femoral component of a hip joint which is intended to be implanted generally in line with the femoral neck axis.

The instrument of the invention has the advantage that disruption to muscle and other soft tissue attached to a bone can be minimised by remote fixation of a location device. In addition, the size of the incision for implantation of the joint prosthesis need not be enlarged relative to the size that is necessary to insert the implant the prosthesis itself, in order to accommodate the instrumentation used to prepare the bone for the implantation.

1. An instrument for use in the orthopaedic surgery for location of a reference point relative to a bone surface, which comprises a plate member having at least two holes in it toward one end for receiving fasteners which can pass through the holes into the bone to fasten the plate member to the bone, a pivot rocker between the said holes which protrudes from the face of the plate member which face the bone surface when the plate member is positioned against a bone, and a reference point towards the opposite end of the plate member from the fastening holes which is spaced from the fastening holes in a direction parallel to the line which intersects the holes, in which the distance between the reference point on the plate member and the surface of the bone can be adjusted by moving the fastener in one of the fastening holes into the bone and moving the fastener in the other of the fastening holes out of the bone.

2. An instrument as claimed in claim 1, which includes a connector rod for another component, which is pivotally connected to the plate member at the reference point.

3. An instrument as claimed in claim 2, in which the connector rod has an arcuate shape so that it can extend around the bone from the bone surface against which the reference point is located to an opposite bone surface to define an axis which extends between the two surfaces.

4. An instrument as claimed in claim 2 or claim 3, in which the connection between the connector rod and the plate member permits relative pivotal motion about at least to axes.

5. An instrument as claimed in claim 4, in which the connector rod includes a mount at its end remote from the connection to the plate member for another instrument component.

6. An instrument as claimed in claim 5, in which the mount for the other instrument component allows the said component to be pivoted relative to the connector rod around an axis which is orthogonal to each of the axes of rotation of the connector rod relative to the plate member.

7. An instrument as claimed in claim 2, in which the connector rod comprises at least two modules, so that the configuration of the connector rod can be changed by replacement of one of the modules.

8. An instrument as claimed in claim 1, in which the plate member has a guide hole in it at the reference point.

9. An instrument as claimed in claim 1, in which the distance between the fastening holes is less than the distance between the reference point and the fastening hole that is nearer to it, measured along the line which intersects the fastening holes or a line extending parallel to it.

10. An instrument as claimed in claim 3, in which the connection between the connector rod and the plate member permits relative pivotal motion about at least two axes.
